(19) United States
(12) Patent Application Publication
(10) Pub. No.: US 2026/0220908 A1
(43) Pub. Date: Jul. 30, 2026

(54) PRIVACY-PRESERVING AI OVER NEXT GENERATION NETWORKS

(71) Applicant: The Trustees of Princeton University, Princeton, NJ (US)

(72) Inventors: Mohamed Seif, Princeton, NJ (US); Andrea J. Goldsmith, Princeton, NJ (US); H. Vincent Poor, Princeton, NJ (US)

(73) Assignee: The Trustees of Princeton University, Princeton, NJ (US)

(21) Appl. No.: 18/921,137

(22) Filed: Oct. 21, 2024

Related U.S. Application Data
(60) Provisional application No. 63/544,870, filed on Oct. 19, 2023.

(51) Int. Cl. G06V 10/72; G06F 21/62; G06V 10/56; G06V 10/764; G06V 10/94; G06V 10/96; G06V 20/54; G06V 40/16; G08G 1/01; G16H 40/67; H04W 16/14

(52) U.S. Cl. CPC G06V 10/72; G06F 21/6245; G06V 10/56; G06V 10/764; G06V 10/95; G06V 10/96; G06V 20/54; G06V 40/168; G06V 40/174; G16H 40/67; H04W 16/14; G06V 2201/08; G08G 1/0125

(57) ABSTRACT

Disclosed is a private collaborative AI-based inference mechanism at the network edge wherein each edge device protects the sensitive information of the extracted features before transmission to the central server for inference. In this setting, the joint inference is divided into three modules: a) sensing for data acquisition, b) feature extraction, and 3) feature encoding for transmission. The techniques broadly include receiving input data representative of a target on a plurality of edge devices. Each edge device may be configured to generate a feature set by extracting one or more features from the input data and removing or obfuscating at least one privacy-sensitive feature of the target. The techniques may include encoding the feature set and transmitting the feature set to one or more remote processors. The remote processor(s) may then infer a classification label by aggregating features sent by each edge device.

### CROSS-REFERENCE TO RELATED APPLICATIONS

This application claims priority to U.S. Provisional Ser. No. 63/544,870, filed Oct. 19, 2023, the contents of which are incorporated by reference herein in its entirety.

### STATEMENT REGARDING FEDERALLY SPONSORED RESEARCH OR DEVELOPMENT

This invention was made with government support under MURI Grant No. FA9550-18-1-0502 awarded by the Air Force Office of Scientific Research (AFOSR). The government has certain rights in the invention.

### TECHNICAL FIELD

The present disclosure relates to privacy-preserving artificial intelligence (AI) over next generation networks.

### BACKGROUND

By way of background, artificial intelligence (AI) is a key technology for next-generation networks; it will empower new functionalities to enable low-latency inference and sensing applications, including autonomous driving, personal identification, and activity classification (to name a few). The disclosed framework provides many use cases over different communication networks (e.g., online social networks, financial networks, indoor Wi-Fi and mobile networks (5G and beyond)). The framework supports a wide range of applications over such networks, including anomaly detection, traffic monitoring, financial trading, portfolio optimization, distributed sensing, object detection, speech recognition, and activity classification.

Prior work has left the design of privacy-preserving mechanisms for AI-based inference over next-generation networks unexplored. Prior work in this area has focused on protecting an individual's privacy while training machine learning models.

In view of prior works, two conventional AI paradigms are commonly used in practice: 1) On-device inference which locally performs AI-based inference, which however suffers from high computation overhead, and 2) On-server inference, where edge devices upload their raw data to a central server to perform a global inference task; however, this framework violates the data privacy of individuals, and it suffers from high communication overhead.

### BRIEF SUMMARY

In various aspects, a method for cooperative inference may be provided. The method may include generating a feature set by receiving input data (such as an image, a video, or an audio recording from one or more recording devices) representative of a target (such as a vehicle or pedestrian on a roadway or sidewalk) on a plurality of edge devices. Each edge device may be configured to extract one or more features from the input data and remove or obfuscate at least one privacy-sensitive feature of the target (such as a facial expression, a hair color, a length of hair, and/or one or more dimensions (of a body or face), etc.). Generating a feature set may include using a pre-trained machine learning model on each edge device to extract one or more initial features from the input data. Generating a feature set may include generating a clipped feature set by subjecting the one or more initial features to controlled clipping using a parameter (C) as a boundary, such that feature elements in the clipped feature set stay within a defined norm range. Generating a feature set may include utilizing noise addition perturbation to protect privacy-sensitive attributes. The input data may include, e.g., video from multiple security cameras.

The method may include encoding the feature set. Encoding the feature set may include compressing the feature set.

The method may include transmitting the feature set to one or more remote processors. The method may include inferring, on the one or more remote processors, a classification label by aggregating features sent by each edge device. Aggregating features may include generating a collective feature set by pooling feature sets, refining the collective feature set, and enhancing inference precision. Pooling feature sets may include utilizing an average function, a maximum function, or a minimum function.

The remote processor(s) may be disposed on a remote server.

The method may include predicting traffic based on the classification label of multiple vehicles. The method may include identifying an anomaly or a security concern based on the classification label from multiple targets. The edge device may be a wearable device on a patient, and the input data may include health-related data. The method may include providing real-time insight into the health of the patient to a healthcare provider. The method may include determining adjustments to lighting, heating, or cooling in a building by aggregating occupancy and sensor data in the building. The method may include adjusting spectrum sharing between wireless networks based on traffic characterization. The method may include determining an object identity based on the aggregated features.

In various aspects, a system may be provided. The system may include one or more edge devices communicatively coupled to an one or more remote processors. Each edge device may be configured to: (i) capture input data representative of a target; (ii) generate a feature set by extracting one or more features from the input data; (iii) generate an obfuscated feature set by removing or obfuscating at least one privacy-sensitive feature of the target from the feature set; (iv) generate an encoded feature set by encoding the obfuscated feature set; and (v) transmit the feature set to one or more remote processors. The one or more remote processors may be configured to, collectively receive each encoded obfuscated feature set, and infer a classification label by aggregating features sent by each edge device.

### BRIEF DESCRIPTION OF THE FIGURES

FIG. 1 is a schematic illustration of a private task-inference framework.

FIG. 2 is a schematic illustration of a holistic system architecture designed to optimized data processing and inference in edge computing environments.

FIG. 3 is a schematic illustration of a collaborative inference pooling operation, where aggregated local feature maps from distributed edge devices contributes to accurate predictions while addressing privacy concerns; the aggregated global map is fed into the model inference for label prediction.

FIG. 4 is a plot showing the impact of perturbation noise on inference accuracy; more feature representation improves the utility-privacy tradeoff, but leads to more latency.

FIG. 5 is a schematic illustration of a private feature aware transmission, where edge device participates in the collaborative inference if the privatized uncertainty score is below a certain threshold.

FIG. 6 is a simplified block diagram of modules on a device.

FIG. 7 is a simplified diagram of a system.

FIG. 8 is a diagram of components in an edge device and edge server.

FIG. 9 is a flowchart of method for using multiple edge devices.

FIG. 10 is a flowchart of method for use with an edge server.

### DETAILED DESCRIPTION

The disclosed process develops new privacy-preserving algorithms for task-oriented inference over communication networks. This requires a new principled design framework that achieves individual privacy.

More particularly, disclosed is a private collaborative AI-based inference mechanism at the network edge wherein each edge device protects the sensitive information of the extracted features before transmission to the central server for inference. In this setting, the joint inference is divided into three modules: a) sensing for data acquisition, b) feature extraction, and 3) feature encoding for transmission. The key design objectives of the disclosed approach are two-fold: 1) minimize the communication overhead, and 2) maintain rigorous privacy guarantees for transmission of features over communication networks while providing satisfactory inference performance.

The disclosed transmission approach is customized to the inference task, unlike previous techniques used for conventional distributed machine learning and communication systems. In this disclosed framework, the joint inference is divided into three modules: a) sensing for data acquisition, b) feature extraction, and 3) feature encoding for transmission. Key design objectives of the disclosed approach include: 1) minimize the communication overhead and 2) maintain rigorous privacy guarantees for the transmission of features over communication networks while providing satisfactory inference performance.

Recent advancements in enhancing data privacy within the context of distributed machine learning can be classified into three principal categories:

Hardware-Based Methods: The first category involves innovative techniques rooted in hardware-based approaches, strategically leveraging trusted execution environments to ensure heightened security.

Cryptographic Methods: The second category centers around cryptographic methodologies, harnessing cutting-edge primitives such as secure multi-party computation, homomorphic encryption, and functional encryption. These cryptographic measures significantly enhance communication security.

Perturbation-Based Strategies: The third class encompasses perturbation-based strategies, characterized by manipulating or regenerating transmitted messages. These strategies incorporate differential privacy (DP) mechanisms, which involve the introduction of Laplacian or Gaussian noise to raw features or model parameters. In alignment with the disclosed approach, presented is a similar perspective by advocating for the introduction of noise within the disclosed approach. This aligns with the third category, where perturbation-based strategies play a significant role in preserving privacy. However, the disclosed approach provides for the introduction of noise, coupled with the utilization of adaptive feature embeddings. This addresses privacy concerns and contributes to preserving utility in the distributed machine-learning context.

Cryptographic Methods vs. Perturbation-Based Methods: In the context of the disclosed approach, it is essential to investigate computational approaches underpinned by cryptographic tools, including but not limited to secure multi-party computation (MPC) and homomorphic encryption. However, it is crucial to recognize that these methods necessitate a meticulous equilibrium between strong security guarantees and the time delay and computational resource demands. Notably, deploying these methods on devices with limited computational capacity, such as the Internet of Things (IoT), presents significant challenges. In contrast, the disclosed approach emphasizes exploring information-theoretic privacy approaches, serving as a more lightweight alternative. These privacy mechanisms aim to establish robust assurances through the incorporation of quantitative information-theoretic principles, effectively achieved through the adept fusion of signal processing and coding techniques. The primary objective is to intricately hide the sensitive information, offering a substantial countermeasure against potential privacy breaches.

Within the data privacy community, privacy-based notions can be classified into two categories: 1) Context-aware notions, which consider the context of the data in the mechanism design, and 2) context-free notions, that is agnostic to the context of any information. Numerous metrics have been proposed to measure context-aware privacy, such as mutual information and maximal leakage. Differential privacy (DP) is the gold standard for providing rigorous privacy guarantees. This notion guarantees that each user's data has a minimal statistical influence on the output of the queries. In the context of the disclosed approach, DP seeks to ensure that the changes in the sensitive attributes do not significantly influence the inference results. This metric has compelling properties, such as the robustness to post-processing that adversaries can perform to distill private information further. Hence, it provides a rigorous barrier to those types of malicious inference.

Within the framework of the disclosed approach, introduced is a comprehensive blueprint for context-free privacy mechanisms. This versatile solution accomplishes two key objectives: 1) It facilitates precise customization of privacy levels, catering to specific needs, and 2) it encourages the development of modular designs characterized by low complexity, particularly suited for deployment on edge devices. This practical implementation entails integrating privacy-preserving mechanisms seamlessly with pre-trained machine-learning models, eliminating the need for extensive model redesign. This augments usability and enhances the feasibility of adopting privacy-preserving strategies in real-world scenarios.

Provided herein is an insightful comparison between the methodology proposed in S. R. Alvar and I. V. Bajic, “Scalable privacy in multi-task image compression,” in Proceedings of the International Conference on Visual Communications and Image Processing (VCIP), pp. 1-5, 2021 and the approach disclosed herein. The authors of the referenced paper introduced a task-aware compression mechanism to safeguard the privacy of sensitive attributes within the observed data. In contrast, the disclosed approach brings forth distinct nuances that set it apart:

Enhanced Privacy Mechanism: The disclosed methodology extends beyond conventional techniques by incorporating an enhanced privacy mechanism, addressing the shortcomings of existing methods.

Multi-Device Scenario: While the referenced authors predominantly considered a single-edge device for feature extraction and processing, the disclosed approach embraces the complexity of multi-device environments, encompassing diverse data sources and processing nodes.

Holistic Task Spectrum: Unlike the singular focus on specific tasks presented in Alvar (2021), the disclosed approach spans a broader array of tasks, catering to the diverse needs of modern data-driven applications.

Privacy-Preserving Task Variation: While the authors in the referenced paper acknowledged the potential privacy violation resulting from tasks such as image retrieval, the disclosed approach diligently integrates privacy preservation across all tasks, ensuring comprehensive protection.

In conclusion, the disclosed approach transcends the boundaries set by Alvar (2021) by introducing an elevated level of privacy preservation, accommodating complex multi-device scenarios, embracing a broader range of tasks, and ensuring a consistent shield against privacy breaches across all task categories.

Addressing Shortcomings in the Privacy Concept:

The privacy concept of Alvar (2021), termed the “privacy fan,” presents several limitations that necessitate a more refined approach. The primary privacy metric employed by the authors hinges on mutual information, a context-aware measure. However, a thorough analysis reveals several shortcomings in their proposed mechanism:

Average-Case Privacy: Using mutual information for privacy assurance results in average-case protection for input data.

Computational Complexity: The estimation of mutual information tends to be computationally intensive. Additionally, the kernel-density method suggested by the authors tends to underestimate privacy leakage, thereby undermining the precision of the privacy guarantee.

Privacy-Compression Tradeoff: A precise characterization of the delicate balance between privacy preservation and data compression is lacking in the presented framework.

Limited Guarantee Scope: The disclosed mechanism fails to provide a formal assurance for downstream tasks that could be executed at the edge server. It exclusively offers protection for specific inference tasks, contingent upon the trustworthiness of the server.

The approach disclosed herein is a differential privacy approach in response to these limitations. Differential privacy offers a robust privacy concept coupled with tangible utility guarantees. The disclosed objective is to enhance the anonymization of facial images, with a specific focus on safeguarding sensitive attributes. Simultaneously, retained is the essential information required for machine learning-based inference tasks, encompassing detection, recognition, and classification.

In conclusion, the disclosed refined approach addresses the inadequacies inherent in the “privacy fan” concept by embracing differential privacy. By prioritizing enhanced protection and utility preservation, established is a more comprehensive framework for privacy in the context of facial image processing and subsequent inference tasks.

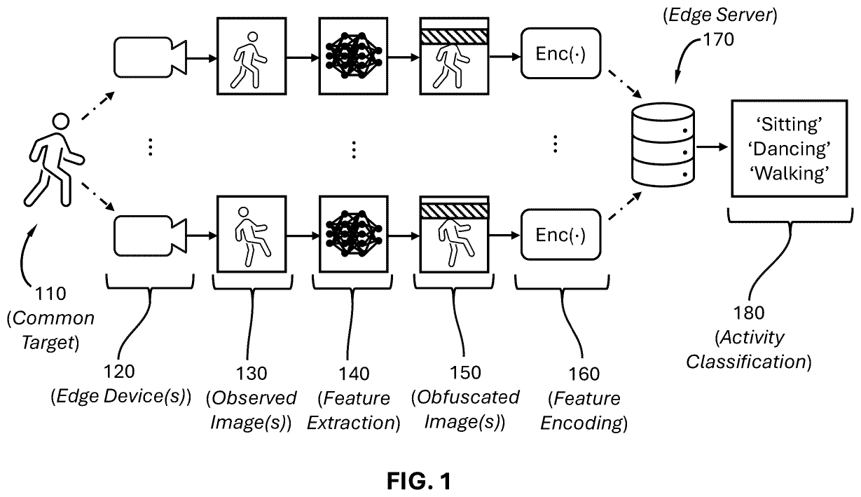

In various aspects, a method for cooperative inference may be provided. Referring to FIG. 1, the method may include one or more processors receiving input data (130) (such as an image, a video, or an audio recording from one or more recording devices) representative of a target (110) (such as a vehicle or pedestrian on a roadway or sidewalk) on a plurality of edge devices (120). The input data my include video from security cameras. The one or more processors maybe within the edge device. Each edge device may be configured to generate a feature set by extracting (140) one or more features from the input data and, and then removing or obfuscating (150) at least one privacy-sensitive feature of the target (such as a facial expression, a hair color, a length of hair, and/or one or more dimensions (of a body or face), etc.).

As used herein, the term “edge device” may refer to a computing device that includes a programmable processor and communications circuitry for establishing communication links to consumer devices (e.g., smartphones, UEs, IoT devices, etc.) and/or to network components in a service provider, core, cloud, or enterprise network. For example, an edge device may include or implement functionality associated any one or all of an access point, gateway, modem, router, network switch, residential gateway, mobile convergence product, networking adapter, customer premise device, multiplexer and/or other similar devices. An edge device may also include various memories and an edge database. Some embodiments may include an edge computing system that includes one or more edge devices, any or all of which may be configured to perform or implement edge computing techniques or technologies.

As used herein, the term “processor” may refer to, is part of, or includes hardware components such as an electronic circuit, a logic circuit, a processor (shared, dedicated, or group) and/or memory (shared, dedicated, or group), an Application Specific Integrated Circuit (ASIC), a field-programmable device (FPD) (e.g., a field-programmable gate array (FPGA), a programmable logic device (PLD), a complex PLD (CPLD), a high-capacity PLD (HCPLD), a structured ASIC, or a programmable SoC), digital signal processors (DSPs), etc., that are configured to provide the described functionality. In some embodiments, the processor may execute one or more software or firmware programs to provide at least some of the described functionality. The term “processor” may also refer to a combination of one or more hardware elements (such as a combination of circuits used in an electrical or electronic system) with the program code used to carry out the functionality of that program code. The term “processor” may also refer to one or more application processors, one or more baseband processors, a physical central processing unit (CPU), a single-core processor, a dual-core processor, a triple-core processor, a quad-core processor, and/or any other device, or combination of devices, capable of executing or otherwise operating (either individually or as a combined unit of processing components), collectively, computer-executable instructions such as program code, software modules, and/or functional processes.

Generating a feature set may include using a pre-trained machine learning model on each edge device to extract one or more initial features from the input data. Any appropriate technique for such feature extraction may be utilized. In some embodiments, generating a feature set may include generating a clipped feature set by subjecting the one or more initial features to controlled clipping using a parameter (C) as a boundary, such that feature elements in the clipped feature set stay within a defined norm range.

Generating a feature set may include utilizing noise addition perturbation to protect privacy-sensitive attributes. The method may include encoding (160) the feature set. Encoding the feature set may include compressing the feature set.

The method may include transmitting the feature set to one or more remote processors (170) (such as remote processor(s) on a remove server). The method may include inferring (180), on the one or more remote processors, a classification label by aggregating features sent by each edge device. Aggregating features may include generating a collective feature set by pooling feature sets, refining the collective feature set, and enhancing inference precision. Pooling feature sets may include utilizing an average function, a maximum function, or a minimum function.

Thus, as seen in FIG. 1, Each edge device (120) extracts features from the observed input that preserves some task-relevant information and satisfies rigorous levels of privacy. Then, each device forwards the processed features over a communication channel to be processed by the server.

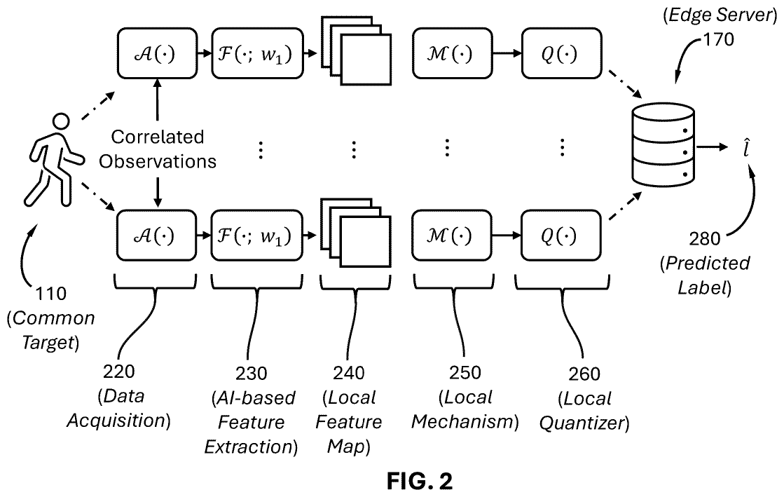

This process is further understood in view of FIG. 2, where a holistic system architecture is described. There, the comprehensive framework encompasses data acquisition (220), feature extraction (230, resulting in local feature maps (240)), manipulation and privacy preservation (mechanism (250) for obscuring certain user information, and a local quantizer (260), and collaborative inference (280). The diagram showcases the orchestrated interplay of components across edge devices and an edge server, enabling efficient and accurate analysis while maintaining data privacy and transmission efficiency.

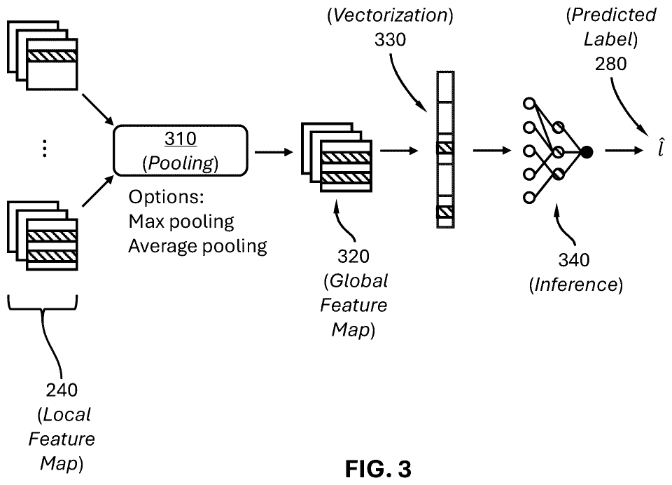

Some collaborative inference pooling operations can be understood with respect to FIG. 3. In FIG. 3, an illustration of the pooling operation employed in one collaborative inference system can be seen. A global feature map (320) formed by aggregating/pooling (310) local feature maps (240) from distributed edge devices contributes to accurately predicted labels (280) while addressing privacy concerns. The aggregated global feature map (320) is vectorized (330) and fed into the model inference (340) for label prediction.

The disclosed technique is highly flexible, and may be utilized for a number of potential privacy-related applications.

For example, the method may include predicting traffic based on the classification label of multiple vehicles. For example, without having received any privacy-related features of images or video recorded by camera(s), a server may utilize a trained model to accurately classify one or more target objects (e.g., vehicles and/or people) in images and/or video (e.g., video or images taken of a highway, people crossing at a crosswalk in an urban area, etc.) In some embodiments, “traffic” can be based on a moving average of a number of detected target objects. With location information and the number of target objects identified, the system can determine one or more characteristics associated with movement of the objects (such as, e.g., flow patterns of vehicles or pedestrians over time at a particular location).

The method may include identifying an anomaly or a security concern based on the classification label from multiple targets. For example, if cameras detect people on an interstate highway, it may be identified as an anomaly or security concern. In some embodiments, if a vehicle is detected as being off the normal path that vehicles take on a given road, it may be identified as an anomaly or security concern. In some embodiments, traffic patterns for a particular location at a particular time of day are outside a predetermined range of variability (e.g., if a residential street is seeing a package delivery truck at 2 am), it may be identified as an anomaly or security concern.

The method may include determining adjustments to lighting, heating, or cooling in a building by aggregating occupancy and sensor data in the building. For example, if the system determines people are only present on the second floor of a three-story building, it may shut off lighting on the first and third floors.

In some embodiments, the edge device may be configured to capture data other than images, video, or audio. For example, the edge device may be a wearable device on a patient, and the input data may include health-related data. In some embodiments, wearable devices and devices to monitor audio and/or visual information may be combined. The method may include providing real-time insight into the health of the patient to a healthcare provider.

Similarly, the edge devices may include, e.g., routers, etc., and the data being captured may related to traffic on the network. The method may include adjusting spectrum sharing between wireless networks based on traffic characterization. For example, based on traffic, the system may be configured to, e.g., throttle one or more users if that.

The method may include determining an object identity based on the aggregated features.

In various aspects, a system may be provided. The system may include one or more edge devices communicatively coupled to an one or more remote processors. Each edge device may be configured to: (i) capture input data representative of a target; (ii) generate a feature set by extracting one or more features from the input data; (iii) generate an obfuscated feature set by removing or obfuscating at least one privacy-sensitive feature of the target from the feature set; (iv) generate an encoded feature set by encoding the obfuscated feature set; and (v) transmit the feature set to one or more remote processors. The one or more remote processors may be configured to, collectively receive each encoded obfuscated feature set, and infer a classification label by aggregating features sent by each edge device.

### EXAMPLES

Disclosed next is a series of examples that demonstrate the innovative potential of the disclosed collaborative inference systems. These systems leverage the power of distributed devices and data aggregation to drive efficiency, accuracy, and real-time insights across diverse domains, from personalized recommendations to disaster response. While showcasing the benefits of collaborative inference, also acknowledged is the importance of addressing privacy concerns associated with data aggregation and analysis. The examples discussed below exemplify the versatility and value of the disclosed collaborative inference systems, each designed to address specific challenges and opportunities in the modern digital landscape.

### Example 1: Traffic Prediction for Smart Cities

Use Case: Collaborative inference systems enhance traffic prediction in smart cities by combining data from various sensors and devices. This optimization reduces congestion and improves overall traffic flow.

Example Privacy Threats Avoided by Using The Disclosed Approach: (i) Location Privacy: Aggregated traffic data could potentially reveal individuals'movement patterns, compromising their location privacy; (ii) Behavior Profiling: Analyzing aggregated traffic data might lead to profiling of individuals'daily routines and habits.

### Example 2: Edge Computing for Real-Time Video Analysis

Use Case: Wireless security cameras collaborate in edge computing to analyze video footage in real-time, identifying anomalies and enhancing security applications.

Example Privacy Threats Avoided by Using The Disclosed Approach: (i) Video Surveillance Intrusion: Collaborative cameras could inadvertently capture private areas, leading to privacy violations; (ii) Unauthorized Recognition: Aggregated video data might be misused for unauthorized facial recognition and tracking.

### Example 3: Healthcare Monitoring and Diagnostics

Use Case: Collaborative inference systems in healthcare monitor patients'vital signs using wearable devices, providing real-time insights to healthcare providers while maintaining data privacy.

Example Privacy Threats Avoided by Using The Disclosed Approach: (i) Medical Data Exposure: Aggregated health data might be intercepted, compromising individuals'medical histories and conditions; (ii) Sensitive Diagnoses: Analysis of collaborative medical data could reveal sensitive health conditions.

### Example 4: Energy Management in Smart Buildings

Use Case: Collaborative inference systems optimize energy consumption by aggregating occupancy and sensor data in smart buildings, enhancing energy efficiency while respecting privacy.

Example Privacy Threats Avoided by Using The Disclosed Approach: (i) Occupancy Profiling: Aggregated occupancy data could lead to profiling of individuals'routines and activities; (ii) Behavior Analysis: Collaborative energy usage data might reveal personal habits and behaviors.

### Example 5: Spectrum Sharing Through 5G-NR Traffic Characterization

Use Case: Collaborative inference systems leveraging 5G NR (New Radio) traffic characterization optimize spectrum sharing between wireless networks, enhancing network performance without compromising privacy.

Example Privacy Threats Avoided by Using The Disclosed Approach: (i) Traffic Analysis: Analyzing NR traffic patterns might lead to inference of user behaviors and preferences; (ii) Network Fingerprints: Collaborative traffic data could inadvertently identify specific users or devices.

### Example 6: Sensing Applications in 6G Systems

Use Case: Collaborative inference systems in 6G networks revolutionize sensing applications by enabling real-time data collection and analysis while safeguarding user privacy.

Example Privacy Threats Avoided by Using The Disclosed Approach: (i) Location Privacy: Highly precise 6G sensing could reveal individuals'precise locations, violating privacy; (ii) Personal Behavior Profiling: Aggregated sensor data might enable profiling of individuals'daily activities and behaviors.

### Example 7: Object Recognition and Multi-Class Classification

Use Case: Collaborative inference systems empower object recognition and multi-class classification by pooling data from diverse sources. These systems enable accurate identification of objects and categorization into multiple classes, enhancing applications such as image processing and automated quality control.

Example Privacy Threats Avoided by Using The Disclosed Approach: (i) Data Leakage: Collaborative model updates could unintentionally reveal specific object instances from various sources; (ii) Unauthorized Class Inference: Attackers might exploit model updates to infer the presence of certain classes within the aggregated data.

### Example 8

Below is an illustrative example to support the disclosed approach: a cooperative inference framework (as shown in FIG. 1) with multiple edge devices performs an AI-based classification task (e.g., activity classification) on a common object (e.g., personal image, voice, or video), where the inference is performed by a central edge server. The inference process can be decomposed into three sub-problems:

Sub-problem 1. Feature extraction aims to obtain an informative representation of the common target (e.g., an image) while removing or obfuscating the sensitive features (e.g., facial expression).

Sub-problem 2. Feature encoding, where the goal is to encode the resulting features for efficient and reliable transmission over the communication channels between the edge devices and the server.

Sub-problem 3. Joint inference, where the central server aggregates the features sent by the edge devices to infer the classification label (i.e., type of activity) of the observed common object.

FIG. 2 outlines a multifaceted framework designed to enhance the efficiency and efficacy of data processing, feature extraction, and inference tasks in an edge computing environment. The system is structured as follows:

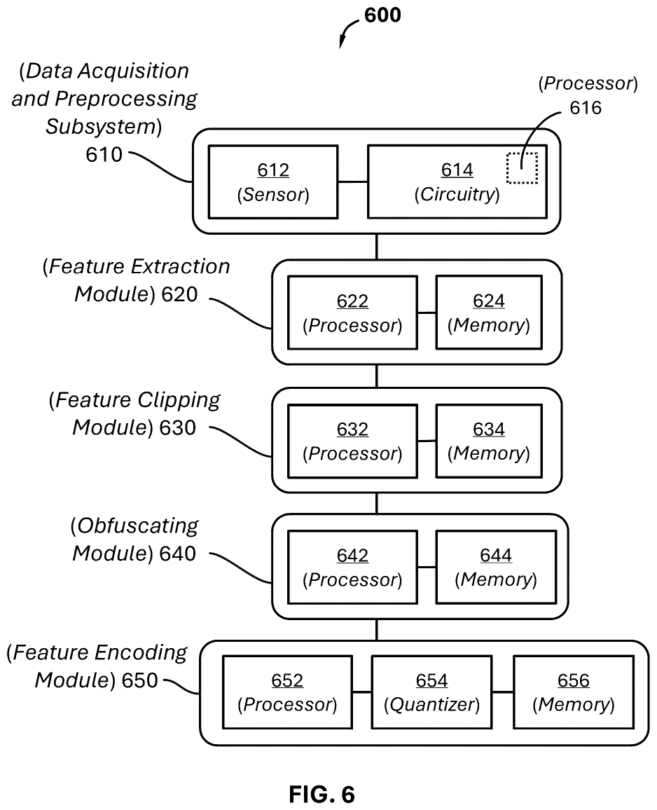

Data Acquisition and Preprocessing Subsystem (FIG. 2, ref. 220): Each edge device is equipped with a data acquisition mechanism tailored for the target object or signal to be classified. This process optimizes the representation of the object or signal, beginning with signal sampling and potentially followed by quantization (FIG. 2, ref. 260) to a finite bit range. Referring briefly to FIG. 6, such a subsystem (610) may include a sensor (612) coupled to circuitry (614). The circuitry (614) may include, e.g., one or more processors (616).

Machine Learning-Powered Feature Extraction Module (FIG. 2, ref. 230): Central to the disclosed technique is using a pre-trained machine learning model on each edge device. This module adeptly extracts vital features from input data, encapsulating essential traits required for downstream analysis. Referring briefly to FIG. 6, such a module (620) may include one or more processors (622), which may be operably coupled to memory (624). As will be understood, in some embodiments, the components that provide this functionality may be a separate module, or may be at least partially integrated into another module. For example, this module may utilize a separate processor, or this may utilize the same processor as in the data acquisition and preprocessing system (processor (616)).

Feature Clipping Mechanism (FIG. 2, ref. 240): The disclosed technique involves applying a feature clipping process. This post-extraction operation subjects the feature map to controlled clipping using a parameter “C” as a boundary. This mechanism ensures that feature elements stay within the defined norm range, aligning with predetermined criteria while preserving key features. Referring briefly to FIG. 6, such a module (630) may include one or more processors (632), which may be operably coupled to memory (634). As will be understood, in some embodiments, the components that provide this functionality may be a separate module, or may be at least partially integrated into another module. For example, this module may utilize a separate processor, or this may utilize the same processor as in the data acquisition and preprocessing system (processor (616)) and/or the feature extraction module (processor (622)).

Sensitive Feature Protection through Obfuscation (FIG. 2, ref. 250): A privacy-preserving mechanism is employed to safeguard against unintended disclosure of sensitive attributes. This mechanism introduces a noise addition perturbation technique to protect attributes like facial expressions. It ensures that modifications to sensitive attributes do not unduly influence inference outcomes while maintaining the overall statistics of the perturbed feature map. Referring briefly to FIG. 6, such a module (640) may include one or more processors (642), which may be operably coupled to memory (644). As will be understood, in some embodiments, the components that provide this functionality may be a separate module, or may be at least partially integrated into another module. For example, this module may utilize a separate processor, or this may utilize the same processor as in the data acquisition and preprocessing system (processor (616)), the feature extraction module (processor (622)), and/or the feature clipping module (processor (632)).

Efficient Feature Encoding for Transmission (FIG. 2, ref. 260): When communication occurs over bandwidth-constrained channels, the edge device employs feature encoding to compress extracted features for streamlined transmission. Quantization discretizes feature map elements into a finite set of bits, enabling efficient transmission without compromising essential information. Referring briefly to FIG. 6, such a module (650) may include one or more processors (652), which may be operably coupled to memory (644). As will be understood, in some embodiments, the components that provide this functionality may be a separate module, or may be at least partially integrated into another module. For example, this module may utilize a separate processor, or this may utilize the same processor as in the data acquisition and preprocessing system (processor (616)), the feature extraction module (processor (622)), the feature clipping module (processor (632)), and/or the obfuscating module (processor (642)).

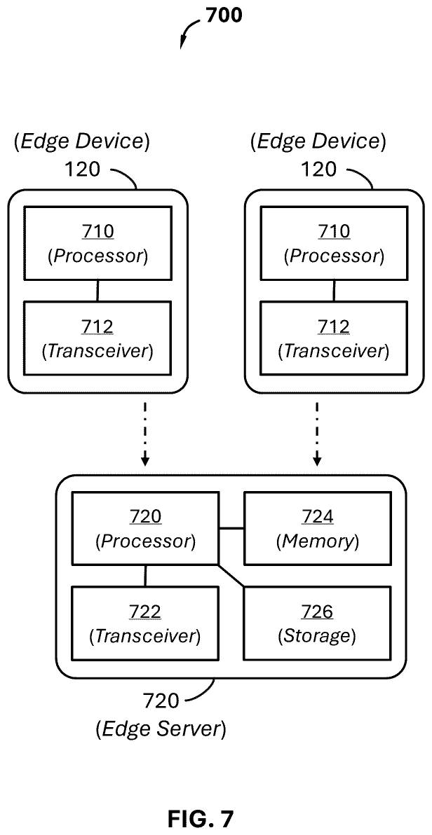

Feature Aggregation and Inference at Edge Server ((FIG. 2, ref. 270): The edge server's convergence point is where features from various edge devices are combined for ML-based inference, such as object classification. Feature aggregation is accomplished through pooling strategies like average pooling and max pooling, refining the collective feature set, and enhancing inference precision. Referring briefly to FIG. 7, an edge server (700) can be seen in communication with multiple edge devices (120) (each edge device having a transceiver (712)). The edge serve may have one or more processors (720), which may be operably coupled to a transceiver (722), memory (724), and/or non-transitory storage devices (726).

FIG. 3 illustrates an exemplary collaborative inference pooling operation employed in the disclosed collaborative inference system. Here, pooling (310) is used to aggregated local feature maps (240) from distributed edge devices, the local maps contributing to accurate predictions while addressing privacy concerns. The aggregated global map (320) may be, e.g., vectorized (330) and fed into the model inference (340), for label prediction (e.g., one or more predicted labels (280) are output).

The disclosed approach is a comprehensive system encompassing data acquisition, machine learning-driven feature extraction, feature manipulation, privacy preservation, efficient data transmission, and collaborative inference. Presented is an innovative framework introducing context-free mechanisms with broad applicability. This framework empowers one to accomplish, inter alia, the following:

Tailored Precision and Customizable Privacy: An important aspect of the disclosed framework lies in its capability to ensure exacting and personalized levels of privacy. This capability is underpinned by the seamless integration of flexible mechanisms adaptable to various scenarios. The disclosed approach empowers one to finely calibrate privacy measures, catering to specific needs and contexts.

Streamlined Modular Designs and Enhanced Edge Device Efficiency: The disclosed framework serves as a catalyst for streamlined modular designs, characterized by their efficiency and simplicity, particularly when deployed on edge devices. Notably, this enables the integration of the disclosed privacy-preserving mechanism in conjunction with pre-trained machine learning models without necessitating a comprehensive re-design process. This integration yields a harmonious coexistence that optimizes resource utilization and augments overall operational efficiency.

Leveraging Correlated Noises for Enhanced Inference Accuracy: The disclosed privacy mechanism leverages the correlation among the inputs of the edge devices, as they collectively capture data from the same object. When the correlation between the inputs of the edge devices is strong, one can harness this correlation by introducing correlated noises. These correlated noises are strategically applied to enhance the accuracy of the model's inference process. By doing so, one can exploit the shared information among the correlated inputs to refine and improve the overall performance of the model's predictions.

Enhancing the Tradeoff between Accuracy and Privacy in Collaborative Inference Systems: Notably, perturbation mechanisms to preserve privacy may deteriorate the inference performance. To counter this, implemented is a straightforward re-transmission strategy to amplify accuracy. This mechanism utilizes a carefully chosen inference threshold denoted as “pth” to control re-transmission termination. When the inferred confidence exceeds the threshold “pth,” the inference process concludes; otherwise, additional edge features are requested, enhancing the inference's accuracy. This adaptable threshold balances inference accuracy and communication overhead, an important factor in resource-limited scenarios. The highest predicted probability is utilized as the disclosed confidence measure, facilitating precise outcomes and efficient re-transmissions. It is worth highlighting that the potential risk of data leakage scales with the number of re-transmissions of private features. This introduces a tradeoff between the accuracy of the inference results and concerns related to data leakage. This tradeoff underscores the significance of meticulously calibrating and optimizing the re-transmission mechanism to achieve the desired equilibrium between precise outcomes and preserving the user's privacy.

Device Selection based on Features Quality: We also introduce a private feature-aware transmission scheme aimed at improving classification accuracy in different data collection scenarios. In these cases, certain features of the data have varying importance regarding privacy and their impact on the final decision. For example, when an edge device captures a noisy image, the device can locally assess whether it should participate less actively in the task based on the quality of the data. This decision-making process is guided by the output of a lightweight classification model, which estimates how confident the device should be in its decision.

Private Feature-Aware Transmission Scheme: This scheme enhances classification accuracy by selectively transmitting data from edge devices based on their local inference, considering the importance of the extracted features in terms of privacy and inference quality. In environments where devices acquire varying quality data, such as noisy images, local inference at each device helps determine whether it should participate in the task.

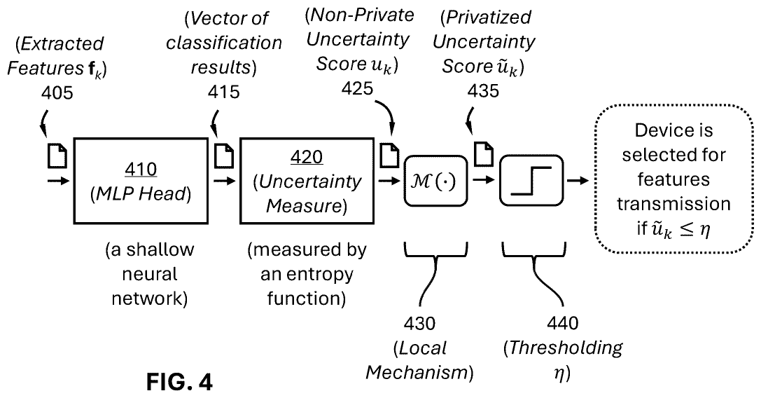

As shown in FIG. 4, each edge device first estimates the confidence of its inference using a lightweight local model. For example, with extracted features fₖ (405) as input, a relatively shallow (e.g., only a few convolution layers, such as 2-4) multilayer perceptron (MLP) (410) may be used to output a vector (415) of classification results. A non-private uncertainty score uₖ (425) based on entropy function (420) is then computed, which reflects the confidence level of the device's inference.

To protect the privacy of the uncertainty scores which are functions of the sensitive features, each device adds privacy-preserving noise (e.g., Gaussian noise) to its uncertainty score, ensuring that the exact value remains confidential, calibrated to meet specific privacy requirements. Thus, a local mechanism (430) may be used to convert the non-private uncertainty score uₖ (425) into a private uncertainty score ũₖ (435).

To minimize unnecessary communication and optimize system performance, the device uses a thresholding (440) process and transmits its features only if the uncertainty score (e.g., private uncertainty score ũₖ (435)) is below a predefined threshold η. This selective mechanism ensures that only confident devices transmit, improving resource efficiency.

In some aspects, one of two participation schemes may be employed.

Local Device Selection: In a first scheme, each device individually decides whether to transmit based on its private uncertainty score. A device transmits its features if the privatized score is below the threshold η. The probability of participation depends on both the original uncertainty score and the noise added for privacy protection.

Server-based Device Selection: In this approach, devices send their privatized uncertainty scores to a central server, which then selects the devices with the lowest scores. This centralized method ensures a more informed and efficient selection of participating devices.

In both cases, differential privacy ensures that the uncertainty score remains private, while the system is still able to make effective decisions about which devices should participate in the task based on the quality of their data.

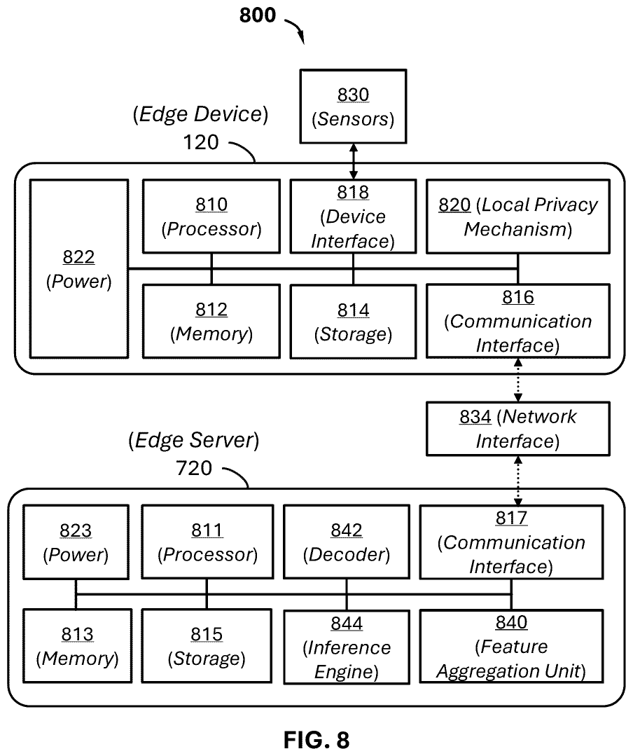

FIG. 8 shows an embodiment of basic elements that may be present in an edge device (120) and edge server (720).

The edge device (120) may include one or more processors (810), which may be, e.g., a CPU, GPU, etc. The one or more processors (810) are generally responsible for executing feature extraction, dimensionality reduction, and noise addition tasks on the collected data. The edge device (120) may include memory (812) (e.g., RAM), to provide temporary storage for holding input data, extracted features, and intermediate results during processing. The edge device (120) may include storage (814), such as a flash or solid state drive (SSD), that may be used for, e.g., storing the pre-trained model and local data before it is processed. It can also store configurations and logs. The edge device (120) may include a communications interface (816), such as a wireless interface, that facilitates communication with the edge server (720) via Wi-Fi, 4G/5G, or any other appropriate communication protocol, preferably a wireless communication protocol. As will be understood, regarding communication protocols, communication between the edge devices (120) and the edge server (720) will typically occur through a network using Wi-Fi, Ethernet, or Cellular Networks (4G/5G). The edge device (120) may include a device interface (818), which may be responsible for gathering observations/data from sensors (830) or other sources like cameras or IoT devices. In some embodiments, such sensors or other sources may be hardwired or electrically coupled to the device interface. The edge device (120) may include a local privacy mechanism (820), A component or software module for implementing the noise addition mechanism for differential privacy (e.g., for incorporating Gaussian Noise Addition). The edge device (120) may include a power supply (822) to provide the necessary energy to power the device and its components.

A network interface (834) (which may be a physical device and/or software-based) may be configured to facilitate data routing from multiple edge devices to a central server (e.g., edge server (720)). It may also provide a feedback loop to send inference results or model updates back to edge devices.

The edge server (720) may include a processor (811), which may be, e.g., a high-performance CPU or GPU, etc. The processor (811) may be configured to performs feature aggregation, model inference, and global decision-making tasks based on the received data. The edge server (720) may include memory (813) (e.g., RAM), to provide temporary storage for pooling feature vectors from all edge devices, decoding feature maps, and running machine learning models. The edge server (720) may include storage (815), such as a solid state drive, and may include one or more databases. The storage may provide persistent storage for storing aggregated data, models, and historical data required for learning and decision-making. The edge server (720) may include a communications interface (e.g., a wireless interface, network interface card, etc.). The communications interface may be configured to receives feature vectors from multiple edge devices (120), typically through a Wi-Fi or Ethernet connection. The edge server (720) may include a feature aggregation unit (840). The feature aggregation unit may be responsible for combining or pooling the feature vectors received from edge devices into a global feature map. The edge server (720) may include a decoder (842), which is a decoding unit or software module that is configured to reconstruct the original features from the global feature map. The edge server (720) may include an inference engine (844) (e.g., a ML model). Preferably, a high-performance module running the machine learning model would be used to predict labels or perform further analysis on the aggregated features. The edge server (720) may include a power supply (823) to provide the necessary energy to power the device and its components.

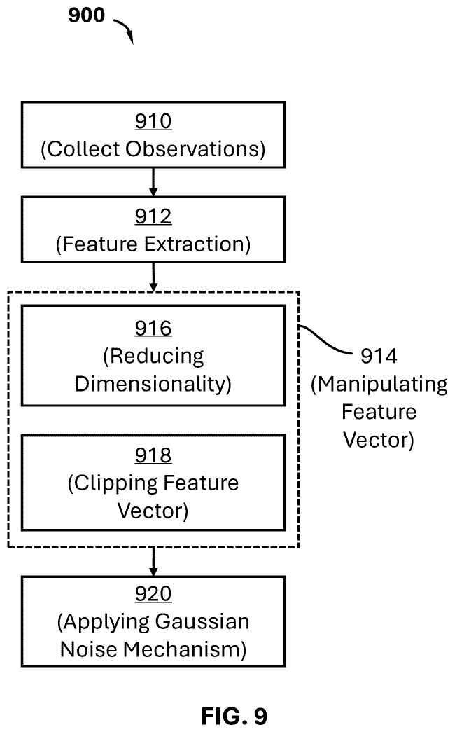

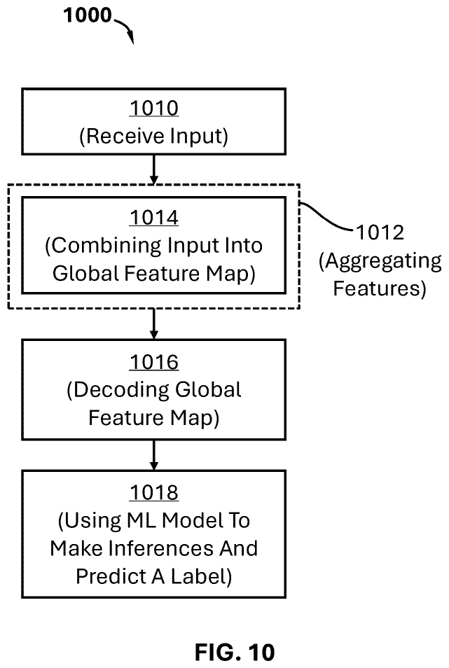

FIGS. 9 and 10 provide flowcharts for an embodiment of how operation of multiple edge devices (FIG. 9) and edge server (FIG. 19) may operate.

As shown in FIG. 9, a flow chart using multiple edge devices is shown. Initially, the method (900) may include having multiple edge devices collect (910) observations. These observations represent data from a target variable or environment. The method may include feature extraction (912). Each edge device processes the observed data in parallel, using a pre-trained model to extract features. The model operates on the data to generate a feature vector.

After feature extraction, each edge device manipulates (914) the feature vector, reduces (914) the dimensionality of the feature vector. This step helps to manage the complexity and size of the data. The resulting feature vector may be clipped (916) to limit its norm to a predefined constant. This may be done to ensure that the data remains within certain bounds, which is important for privacy. Each edge device may then apply (920) a Gaussian noise mechanism to the clipped feature vector. A weight coefficient is used to scale the feature vector, and noise is added to preserve privacy. Such a step may ensure differential privacy by perturbing the feature vector before sharing it. The final output consists of the perturbed feature vectors from all edge devices. Such vectors are the result of the privacy-preserving process, and can be used for further analysis or model training.

Referring to FIG. 10, for the edge server, the method (1000) generally begins with receiving (1010) input. Specifically, receiving as input the perturbed feature vectors from the edge devices. The server then aggregates (1012) the received features. This may be include combining (1014) input into a single global feature map by performing a pooling operation The global feature map is then decoded (1016) using a decoding matrix to reconstruct the original features, forming a decoded global feature map. The decoded global feature map is fed into a ML model at the server, using (1018) a pre-trained model to make inferences and predict a label based on the features. The output is the predicted label.

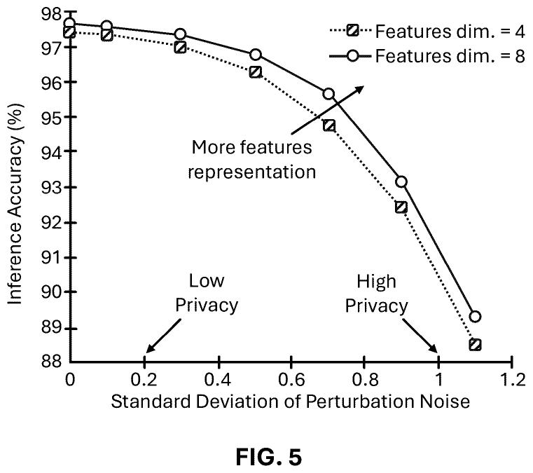

A simple proof-of-concept study of this approach was completed. Considered was an inference system with two edge devices and one edge server for performing two-view image classification based on the MNIST dataset. More specifically, the first edge device observes the first half of an image (“view 1”) while the second device observes the second half (“view 2”). Subsequently, the observed image was perturbed via additive noise drawn from a Gaussian distribution to maintain privacy so that an adversary cannot learn the specific features of the raw data (i.e., the image in the disclosed setup) from the released output. Then performed was feature extraction via a trained neural network to the perturbed image to distill the task-relevant information from this image while preserving the targeted privacy level. Then the extracted feature was encoded as bit sequences for reliable and low-latency transmission over a network. FIG. 5 shows the tradeoff between inference accuracy and the amount of noise used for image privacy, where the privacy levels are measured precisely by the notion of differential privacy (DP). DP ensures that the changes in the raw data (i.e., the image-sensitive features) do not significantly change the statistics of the activity classification decisions and hence limit the inference of any specific entry of the image that any untrusted party can perform on the observed inference results. The features to be sent to the edge server were represented by vectors, each of dimensions four and eight, respectively. A specific resolution represents each element in this vector as a sequence of five bits.

It is envisioned that the disclosed privacy-preserving task-oriented inference framework for the next generation networks will reduce communication overhead and maintain strong privacy guarantees. The disclosed framework provides many use cases over different communication networks (e.g., online social networks, financial networks, indoor Wi-Fi and mobile networks (5G and beyond)). The framework supports a wide range of applications over such networks, including anomaly detection, traffic monitoring, financial trading, portfolio optimization, distributed sensing, object detection, speech recognition, and activity classification.

What is claimed:

1. A method for cooperative inference, comprising:
receiving input data representative of a target on a plurality of edge devices, each edge device configured to generate a feature set by extracting one or more features from the input data and removing or obfuscating at least one privacy-sensitive feature of the target;
encoding the feature set;
transmitting the feature set to one or more remote processors; and
inferring, on the one or more remote processors, a classification label by aggregating features sent by each edge device.

2. The method of claim 1, wherein the one or more remote processors are disposed on a remote server.

3. The method of claim 1, wherein generating a feature set includes using a pre-trained machine learning model on each edge device to extract one or more initial features from the input data.

4. The method of claim 3, wherein generating a feature set includes generating a clipped feature set by subjecting the one or more initial features to controlled clipping using a parameter (C) as a boundary, such that feature elements in the clipped feature set stay within a defined norm range.

5. The method of claim 4, wherein generating a feature set includes utilizing noise addition perturbation to protect privacy-sensitive attributes.

6. The method of claim 5, wherein the input data is an image, a video, or an audio recording.

7. The method of claim 6, wherein the privacy-sensitive attributes include a facial expression of the target, a hair color of the target, a length of hair of the target, one or more dimensions of a face of the target, or a combination thereof.

8. The method of claim 1, wherein encoding the feature set includes compressing the feature set.

9. The method of claim 1, wherein aggregating features includes generating a collective feature set by pooling feature sets, refining the collective feature set, and enhancing inference precision.

10. The method of claim 9, wherein pooling feature sets includes utilizing an average function, a maximum function, or a minimum function.

11. The method of claim 1, wherein the target is a vehicle or pedestrian on a roadway or sidewalk.

12. The method of claim 11, further comprising predicting traffic based on the classification label of multiple vehicles.

13. The method of claim 1, wherein at least plurality of edge devices includes a plurality of security cameras.

14. The method of claim 13, further comprising identifying an anomaly or a security concern based on the classification label from multiple targets.

15. The method of claim 1, wherein the edge device is a wearable device on a patient, and the input data includes health-related data.

16. The method of claim 15, further comprising providing real-time insight into health of the patient to a healthcare provider.

17. The method of claim 1, further comprising determining adjustments to lighting, heating, or cooling in a building by aggregating occupancy and sensor data in the building.

18. The method of claim 1, further comprising adjusting spectrum sharing between wireless networks based on traffic characterization.

19. The method of claim 1, further comprising determining an object identity based on the aggregated features.

20. A system, comprising:
one or more edge devices communicatively coupled to one or more remote processors;
wherein each edge device is configured to:
capture input data representative of a target;
generate a feature set by extracting one or more features from the input data;
generate an obfuscated feature set by removing or obfuscating at least one privacy-sensitive feature of the target from the feature set;
generate an encoded feature set by encoding the obfuscated feature set; and
transmit the feature set to one or more remote processors; and
wherein the one or more remote processors are configured to, collectively:
receive the encoded obfuscated feature set; and
infer a classification label by aggregating features sent by each edge device.
